(19) United States
(12) Patent Application Publication
(10) Pub. No.: US 2026/0062762 A1
(43) Pub. Date: Mar. 5, 2026

(54) IDENTIFYING BREEDING CONDITIONS OF LIVESTOCK USING EPIGENETICS

(71) Applicant: Evonik Operations GmbH, Essen (DE)

(72) Inventors: Rose WHELAN, Ambrosden Oxfordshire (GB); Sanjanaa NAGARAJAN, Singapore (SG); Suki ROY, Sydney, New South Wales (AU); Florian BÖHL, Neckargemiind (DE); Kit Yeng WONG, Singapore (SG); Lingzhi HUANG, Singapore (SG); Sarah CHAN, Bellevue, WA (US); Daniel FRANKE, Essen (DE)

(73) Assignee: Evonik Operations GmbH, Essen (DE)

(21) Appl. No.: 19/107,274

(22) PCT Filed: Aug. 23, 2023
(86) PCT No.: PCT/EP2023/073127

§ 371 Date: Feb. 27, 2025

(30) Foreign Application Priority Data
Sep. 1, 2022 (EP) 22193448.2

(51) Int. Cl. C12Q 1/6888

(52) U.S. Cl. CPC C12Q 1/6888; C12Q 2600/154

(57) ABSTRACT

The present invention relates to a method of certifying a test animal-derived product sample, the method comprising the steps of:
(a) determining a test methylation profile of one or more pre-selected methylation sites within the genomic material obtained from the test animal derived product sample; and
(b) comparing the test methylation profile obtained from (a) with a reference methylation profile obtained from a control animal of the same biological taxon of the test animal from which the product sample is derived from, where the control animal is bred under a known distinct type of animal husbandry;
wherein a significant similarity in the test methylation profile of (a) compared to the reference methylation profile from the control animal, is indicative of the test animal having been bred under the same distinct type of animal husbandry as the control animal and the test animal-derived product is certified so;
wherein a difference in the test methylation profile of (a) compared to the reference methylation profile of the control animal, is indicative of the test animal having been bred under another distinct type of animal husbandry as the control animal; and
wherein the pre-selected methylation sites are CpG sites selected from genes or regions of genomic DNA from the control and test animal that show the highest degree of methylation variation during the training of the method; and
wherein the test methylation profile has significant similarity to the reference methylation profile when the test methylation profile overlaps with the reference methylation profile that is defined by multiple training samples using Principal Component analysis and/or Multi-Dimensional Scaling; and
wherein the methylation profiles are determined using at least one method selected from the group consisting of PCR, methylation-specific PCR, real-time methylation-specific PCR, PCR assay using a methylation DNA-specific binding protein, quantitative PCR, a DNA chip-based assay, pyrosequencing, bisulfate pyrosequencing, Methylated DNA immunoprecipitation-sequencing and combinations thereof; andwherein the test animal is selected from livestock or poultry.

### CROSS-REFERENCE TO RELATED APPLICATIONS

This is a National Stage of International Application No. PCT/EP2023/073127 filed Aug. 23, 2023, claiming priority based on European Patent Application No. 22193448.2 filed Sep. 1, 2022.

### FIELD OF THE INVENTION

The present invention relates to a method of determining the conditions under which an unknown animal derived product sample was bred based on specific panels of CpG sites that provide a source for the generation of DNA methylation profiles which are specific for a distinct breeding condition. In particular, DNA methylation profiling may be used to determine the distinct breeding conditions of animal-derived products. The distinct breeding conditions may include organic, conventional breeding, and the like and may vary based on country and animal type.

### BACKGROUND OF THE INVENTION

There is significant variation in the environmental rearing conditions of agriculturally relevant animals. The conditions an animal is raised in can also affect the outcome of meat characteristics as well as the value of meat as it aligns with customer values. For example, some consumers prefer organic reared chicken meat as it is thought that the rearing standards for organic production enhance the welfare of the birds. Another example is consumer interest in whether the fish they purchased is farmed or wild catch and also in relation to farmed fish, to ensure reduced escapes that may interfere with wild populations.

Currently, the assessment of meat products to categorize them based on rearing environment is done via auditing which may or may not be supported by artificial intelligence and/or block chain technologies. However, most assessments still rely on data recording. In some scenarios genetic testing may be utilized if the strain and therefore genetic background of animals used in different rearing categories differ. For example, the slow-growing strains of broiler chickens differ from the ultra-high growth efficiency strains used in conventional production.

However, the genetic testing of animals would not be sufficient to assess whether the specific strains of animals were indeed reared in the environments that categorizes them as conventional, organic, farmed or wild catch. For example, if a slow-growing strain of broilers chickens were reared without the reduced stocking densities, access to outdoors, enrichments or non-GMO feeds, a genetic test would not be able to determine this. Likewise, if farmed fish strains escape and are later caught by commercial fishing genetic testing alone would misclassify those fish. Therefore, there is still a need for scientific assessments of meat products that ensure appropriate categorizations based on environmental rearing conditions.

Epigenetics is the study of inherited traits caused by mechanisms other than changes in the underlying DNA sequence. In other words, epigenetic marks “orchestrate” our genes. Epigenetic marks can be either chemical (e.g. methylation), protein-based (e.g. histones) or a combination of the two. During development and cell differentiation, DNA methylation is dynamic, but some DNA methylation patterns may be retained as a form of epigenetic memory, accumulated and/or inherited to next generation. Those changes might be responsible for heritable changes in gene activity as DNA methylation events have been shown to be regulation mechanisms associated with gene silencing, expression, chromatin remodelling or imprinting. Epigenetics is attractive for animal breeding as it may identify causality and heritability of complex traits and diseases. DNA methylation patterns are modified along the life of an individual by environmental forces like diet, stress, drugs, or pollution among many others. Some environments are more likely to increase certain methylation patterns, and these patterns could contribute to the epigenetic and/or phenotypic variation between individuals.

Recent studies have shown that DNA methylation patterns of animals contain important information about their rearing conditions. For example, a comparison of genome-wide patterns of methylation and variation at the DNA level revealed that a highly significant proportion of epigenetic variation could be associated with fitness and environmental differences such as captivity in salmon (Le Luyer J et al. 2017 PNAS vol 114, no 49).

Koop et al. and Rhein et al. also separately confirmed that methylation patterns are maintained postmortem and are consistent regardless of the level of decomposition of the sample. One of the only aspects that still affects the methylation pattern of the sample would be the DNA integrity and amount of DNA that is found in the sample (Koop et al., 2021 International Journal of Legal Medicine 135:167-173 and Rhein et al., 2015 Frontiers in Genetics 6:182).

In view of the above, there is still an urgent need to provide a means possibly using epigenetics for identifying and controlling the labelling of animal derived products from animals reared under different conditions, in particular food and more particularly animal material derived to satisfy living standards and eating choices made by consumers.

### BRIEF DESCRIPTION OF FIGURES

FIG. 1 is Principle Component Analysis (PCA) of CpG sites with min coverage 10 in all samples: 6458063 CpG sites

FIG. 2 is Principle Component Analysis (PCA) of 201246 Differentially methylated positions (DMPs) identified.

### DESCRIPTION OF THE INVENTION

The present invention attempts to solve the problems above by providing a method using DNA methylation patterns to distinguish one type of animal derived product from another type of animal derived product and to be able to determine the means and environment of rearing the animal from which the animal derived product derives from and thereby certify the product accurately and reliably. The present invention is based on the finding that the means of and environment of rearing can permanently change the genome of the animal through epigenetics. In particular, the capability to adapt to the environment and maintain the adapted biological pattern depends on epigenetic mechanisms, including DNA methylation. In particular, the present invention is based on the finding that the method and environment of rearing an animal for food consumption may also result in changes in epigenetic mechanisms of the animal, including DNA methylation patterns and these patterns may be passed down to the different products that may derive from the animal.

The inventors have unexpectedly found that this property can be utilized to identify “epigenetic fingerprints” on the genome that are specific to a method and environment of rearing of not just one animal but possibly all the animals that undergo the same method and environment of rearing. Based on these findings, the present invention provides means to identify the specific environment or breeding conditions that an animal was put through, particularly rearing animals also known as livestock and poultry from which an animal-derived product comes from. In particular, the method according to any aspect of the present invention may be used to determine if an animal-derived product is derived from an animal that has been reared under distinct conditions or not. More in particular, the method according to any aspect of the present invention may be used to determine if an animal-derived product is derived from an animal that has been bred under distinct animal husbandry conditions. In this way, the method according to any aspect of the present invention may then be used to accurately and reliably determine the specific animal husbandry conditions under which an animal was bred and then provide and/or confirm the certification of any sample that originates from the animal. Further, the method according to any aspect of the present invention may also be used to identify if any animal-derived product is accurately and reliably certified, particularly where the certification is based on the breeding conditions of the animal.

According to one aspect of the present invention, there is provided a method of certifying a test animal-derived product sample, the method comprising the steps of:
(a) determining a test methylation profile of one or more pre-selected methylation sites within the genomic material obtained from the test animal derived product sample; and
(b) comparing the test methylation profile obtained from (a) with a reference methylation profile obtained from a control animal of the same biological taxon of the test animal from which the product sample is derived from, where the control animal is bred under a known distinct type of animal husbandry,
wherein a significant similarity in the test methylation profile of (a) compared to the reference methylation profile from the control animal, is indicative of the test animal having been bred under the same distinct type of animal husbandry as the control animal and the test animal-derived product is certified so; and
wherein a difference in the test methylation profile of (a) compared to the reference methylation profile of the control animal, is indicative of the test animal having been bred under another distinct type of animal husbandry as the control animal;
wherein the pre-selected methylation sites are CpG sites selected from genes or regions of genomic DNA from the control and test animal that show the highest degree of methylation variation during the training of the method; and
wherein the test methylation profile has significant similarity to the reference methylation profile when the test methylation profile overlaps with the reference methylation profile that is defined by multiple training samples using Principal Component analysis and/or Multi-Dimensional Scaling; and
wherein the methylation profiles are determined using at least one method selected from the group consisting of PCR, methylation-specific PCR, real-time methylation-specific PCR, PCR assay using a methylation DNA-specific binding protein, quantitative PCR, a DNA chip-based assay, pyrosequencing, bisulfate pyrosequencing, Methylated DNA immunoprecipitation-sequencing and combinations thereof; and
wherein the test animal is selected from livestock or poultry.

In particular, in step (b) of the method according to any aspect of the present invention the test methylation profile determined in (a) is compared with a panel of predetermined reference methylation profiles of the same biological taxon of the test animal from which the product sample derives, wherein each of the predetermined reference methylation profiles is specific for a control animal that has been bred under a known distinct type of animal husbandry, wherein if the test methylation profile is significantly similar to one of the predetermined reference methylation profiles, the test animal derived product sample has the same distinct certification as that of the control animal from which the predetermined reference methylation profile was obtained and the test animal-derived product is certified so.

According to another aspect of the present invention, there is provided a method of certifying a test animal-derived product sample, the method comprising the steps of:
(a) determining the test methylation profile of one or more pre-selected methylation sites within the genomic material obtained from the test animal derived product sample; and
(b) comparing the test methylation profile determined in (a) with a panel of predetermined reference methylation profiles of the same biological taxon of the test animal from which the product sample derives, wherein each of the predetermined reference methylation profiles is specific for a control animal that has been bred under a known distinct type of animal husbandry,
wherein if the test methylation profile is significantly similar to one of the predetermined reference methylation profiles, the test animal derived product sample has the same distinct certification as that of the control animal from which the predetermined reference methylation profile was obtained and the test animal-derived product is certified so; and
wherein the test animal is selected from livestock or poultry.

According to yet another aspect of the present invention, there is provided a method of certifying a test animal-derived product sample, the method comprising the steps of:
(a) determining the methylation status of at least one CpG site within the genomic material obtained from the animal-derived product sample,
(b) comparing the methylation status of the CpG site from (a) with that of a control animal of the same biological taxon of the test animal from which the product sample is derived from, where the control animal has been bred under a known distinct type of animal husbandry,
wherein a significant similarity in the test methylation status of (a) in the test animal compared to the CpG site in the control animal is indicative of the test animal having been been bred under the same distinct type of animal husbandry as the control animal and the test animal-derived product is certified so; and wherein a difference in the test methylation status of (a) compared to the to the CpG site in the control animal, is indicative of the test animal having been bred under another distinct type of animal husbandry as the control animal; and wherein the test animal is selected from livestock or poultry.

As used herein, the term ‘animal-derived product’ refers to products that originate from animals. In particular, the term ‘test animal-derived product’ refers to the sample or subject in question that is to be introduced to the array according to any aspect of the present invention. These products from animals may include meat and meat products, also including fat, flesh, blood, processed meat, and lesser-known products, such as isinglass and rennet, poultry products (meat and eggs), dairy products (milk and cheese), and non-food products such as fibre (wool, mohair, cashmere, leather, and the like). Animal-derived products may also include products that can be made using animal products (e.g., fat) such as soap, creams, and such. In one example, the animal-derived product is meat, eggs, blood, brain, sperm, milk and any other tissue or sample that provides genomic DNA. In particular, the animal-derived product is meat. In one example, the animal-derived product sample may be a single type of meat, different types of meat, a single part of a type of meat, different parts of a single type of meat or different parts of different types of meat. In the event the animal is an aquatic animal, these products from animals may include meat and meat products, also including eggs, fat, flesh, blood, processed meat and lesser-known products, and non-food products such as fibre (shells, scales and the like). Animal-derived products may also include products that can be made using animal products (e.g. fish oil) such as tablets, powder and such. In one example, the animal-derived product is meat, eggs, blood, brain, shell, scale, skin, tissue, abdominal muscle tissue or any other tissue or sample that provides genomic DNA. In particular, the animal-derived product is meat, skin, blood, trimmings or any organ from the aquatic animal. In particular, trimmings are used as biproducts for fish meal/oil which end up in the animal feed industry or pets. The sample may be from any biological entity having a DNA genome and DNA genome methylation. In particular, the methylation site is a CpG site.

The term ‘certification’ refers to a certificate or a confirmation given by designated certification agencies that endorse the quality of a particular animal derived product, including food for use and/or consumption by human beings. The term ‘certification of quality’ is used interchangeably with the term ‘certification’. These certifications are usually found on the packaging of the animal-derived product including food to be consumed and are printed by the manufactures of the products. Examples of certifications of distinct food quality may include ‘Haltungsform’, ‘Tierwohl’, ‘Ohne Gentechnik’, ‘halal’, ‘kosher’, ‘organic’, free range’, ‘pasture raised’, ‘grass fed’, ‘grain fed’, ‘vegetarian’, ‘raised without hormones’, and the like. There are different certifications based on the country as well. For example, like Haltungsform in Germany, other certifications include Red Tractor (UK), Label Rouge (France), USDA Grade (USA) etc., and other safe labels that confirm that a product sold has been prepared in accordance with specific religious or safety regulations. Specifically, the term ‘certification’ herein refers to a certificate or a confirmation given by designated certification agencies that endorse the source, quality and/or means of breeding or animal husbandry techniques undergone by an animal from which a particular product is derived from for consumption or use by human beings. According to any aspect of the present invention, the certification of a ‘distinct type of animal husbandry’ refers to a distinct and industrially accepted method of breeding an animal.

In one example, the distinct certification or certification of sample X may be based on a type of animal husbandry that the test animal was reared under. In Germany, this is labelled as ‘Haltungsform’. There are at least four types/conditions under which the animals may be reared. These four levels of animal husbandry include Stable housing (Stallhaltung), Stable housing Plus (StallhaltungPlus), Outside climate (Auβenklima) and Premium (Premium), these are also known as Haltungsform 1, 2, 3 and 4 respectively. Animal products derived from animals bred under different animal husbandry conditions may result in a different DNA methylation profile. The distinct type of animal husbandry may vary depending on the country where the method according to any aspect of the present invention is carried out. Regardless of different terminology used in different countries to describe different distinct animal husbandry practices, the overall concept of the method according to any aspect of the present invention is the same and applicable in any one of these countries

For example, in Germany, the different distinct types of animal husbandry techniques practiced on livestock and poultry may be labelled ‘Haltungsform’ and as mentioned above, are officially and accepted by the industry to be divided into least four types/conditions under which the animals may be reared. These four levels of animal husbandry include Stable housing (Stallhaltung), Stable housing Plus (StallhaltungPlus), Outside climate (Auβenklima) and Premium (Premium). Similarly, in France the livestock and poultry may be labelled ‘label rouge’, ‘organic’, or with other pictograms that display the farming methods the animal went through before the animal derived product was obtained. In the United Kingdom livestock and poultry, the Red Tractor Food Assurance certification scheme exists which includes at least three levels of animal husbandry including Certified Standards, Enhanced Welfare and Free Range. Other labels existing in the United Kingdom include RSPCA Assured which certify specific animal welfare standards and several organic meat certifying schemes such as the Organic Farmers and Growers Certification and the Soil Association Organic Standard. Examples of meat certification in the United States of America (USA) includes those provided by the United States Department of Agriculture (USDA), which include Grade A Carcass Quality and Organic certifications as examples. The USDA also approves some third-party certification schemes such as provided by the nonprofit A Greener World, which include Certified Animal Welfare Approved defining husbandry related to animal welfare and Certified Grassfed defining specific feed types in animal husbandry. In particular, the distinct types of animal husbandry may be selected from the group consisting of:
Certified Standards, Stable housing (Stallhaltung), or equivalents thereof;
Enhanced Welfare, Stable housing Plus (StallhaltungPlus) or equivalents thereof;
Free Range, Outside climate (Auβenklima), Premium (Premium) or equivalents thereof; and
organic.

The term ‘equivalents’ as used herein refers to different terminology for the same or similar distinct type of animal husbandry that is used in different countries. For example, the conditions of breeding an animal under the distinct type of animal husbandry under ‘Certified Standards’ in UK may be the same or at least significantly similar as the conditions of breeding an animal in Germany under Stable housing (Stallhaltung). The same conditions of for this distinct type of animal husbandry may be present in another country under a different term. ‘Equivalents’ thus refers to the same or significantly similar breeding conditions practiced in different countries with different names or certifications.

The term “test” used in conjunction with the term subject and/or animal herein refers to an entity that is subjected to the method according to any aspect of the present invention and is the basis for an analysis application of the present invention. An “(individual) test subject”, an “(individual) group of test subjects” or a “test profile” or an ‘test animal derived product’ is therefore a (individual) subject or group of subjects being tested according to the invention or a profile being obtained or generated in this context. Conversely, the term “reference” shall denote, mostly predetermined, entities which are used for a comparison with the test entity. For example, the term ‘reference animal’ refers to an animal used for comparison or as a control in reference to the ‘test animal’. Similarly, the term ‘sample’ and/or ‘test animal-derived product sample’ used in accordance with any aspect of the present invention refers to an entity that may be subject to the method of the present invention. In particular, a sample may be any (test) animal-derived product that may be subject to the method of the present invention to determine the distinct certification by first determining the DNA methylation profile and then comparing this test methylation profile with a control. Blockchain may also be used to make the information easily available for the consumer.

The method according to any aspect of the present invention may be used to identify an unknown sample (i.e. animal-derived product sample) based on DNA methylation patterns. These DNA methylation patterns may then be compared with reference DNA methylation patterns to trace the animal-derived product sample back to the slaughterhouse or farmhouse from which the sample originates and then determine whether the unknown sample corresponds to an animal which has been bred according to any one of the animal husbandry techniques in question. In this way, a buyer or a consumer of meat can verify that meat being sold as or marketed as being organic or non-organic for example is genuine.

The livestock according to any aspect of the present invention includes terrestrial and aquatic livestock. In particular, livestock may be rearing animals selected from terrestrial and aquatic livestock or poultry. In particular, terrestrial livestock may include cattle, sheep, pigs, goats, horses, camels, donkeys, mules, rabbits and the like and poultry may include chickens, turkeys and other gallinaceous birds, ducks, geese, quail, and the like. As used herein, the term ‘livestock’ may also include poultry and refer to any farm animal or animal that may be used in agriculture.

As used herein, the term “aquatic livestock” refers to any organism that is reared entirely in water or that lives predominantly in water, especially compared with terrestrial animals. These aquatic livestock may live in different water forms, such as seas, oceans, rivers, lakes, ponds, etc. More in particular, the aquatic livestock according to any aspect of the present invention may be may any fish, cephalopod, aquatic molluscs, or aquatic crustaceans, at all life stages, including eggs, sperm and gametes. Even more in particular, the ‘aquatic animal’ means animals of the following species: (i) fish belonging to the superclass Agnatha and to the classes Chondrichthyes, Sarcopterygii and Actinopterygii, (ii) aquatic molluscs belonging to the phylum Mollusca; and (iii) aquatic crustaceans belonging to the subphylum Crustacea. Even more in particular, the aquatic livestock according to any aspect of the present invention may be aquatic livestock used in aquaculture. Some non-limiting examples of aquatic animals according to any aspect of the present invention include barramundi, carp, catfish, halibut, marbled crayfish, marine and brackish fishes, marine shrimp, mitten crabs, mussels, oysters, pangasius, rainbow trout, salmonids, scallops, sea bass, sea bream, soft-shelled crabs, soft-shelled turtles, tiger prawns, tilapia, turbot, white-leg prawn, shrimp, octopus, squid and other decapod crustaceans, bivalves and gastropods.

As used herein, the term “comprising” is to be construed as encompassing both “including” and “consisting of”, both meanings being specifically intended, and hence individually disclosed aspects of the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

In context of the present invention, the terms “methylation profile”, “methylation pattern”, “methylation state” or “methylation status,” are used herein to describe the state, situation or condition of methylation of a genomic sequence, and such terms refer to the characteristics of a DNA segment at a particular genomic locus in relation to methylation. Such characteristics include, but are not limited to, whether any of the cytosine (C) residues within this DNA sequence are methylated, location of methylated C residue(s), percentage of methylated C at any particular stretch of residues, and allelic differences in methylation due to, e.g., difference in the origin of the alleles.

The term “methylation status” refers to the status of a specific methylation site (i.e. methylated vs. non-methylated) which means a residue or methylation site is methylated or not methylated. Then, based on the methylation status of one or more methylation sites, a methylation profile may be determined. Accordingly, the term “methylation profile” or also “methylation pattern” refers to the relative or absolute concentration of methylated C residues or unmethylated C residues at any particular stretch of residues in the genomic material of a biological sample. For example, if cytosine (C) residue(s) not typically methylated within a DNA sequence are methylated, it may be referred to as “hypermethylated”; whereas if cytosine (C) residue(s) typically methylated within a DNA sequence are not methylated, it may be referred to as “hypomethylated”. Likewise, if the cytosine (C) residue(s) within a DNA sequence (e.g., the DNA from a sample nucleic acid from a test subject) are methylated as compared to another sequence from a different region or from a different individual (e.g., relative to normal nucleic acid or to the standard nucleic acid of the reference sequence), that sequence is considered hypermethylated compared to the other sequence. Alternatively, if the cytosine (C) residue(s) within a DNA sequence are not methylated as compared to another sequence from a different region or from a different individual, that sequence is considered hypomethylated compared to the other sequence. These sequences are said to be “differentially methylated”. Measurement of the levels of differential methylation may be done by a variety of ways known to those skilled in the art. One method is to measure the methylation level of individual interrogated CpG sites determined by the bisulfite sequencing method, as a non-limiting example.

The term “hypermethylation” refers to the average methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample. In particular, control refers to an animal derived product that has been obtained from a control animal bred according to a known distinct type of animal husbandry or farming method. A known distinct type of animal husbandry refers to a farming method practiced on the control animal where the farming method was known and confirmed before the animal derived product was obtained from the control animal.

The term “hypomethylation” refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample. In particular, control refers to an animal derived product that has been obtained from a control animal bred according to a known distinct type of animal husbandry or farming method. A known distinct type of animal husbandry refers to a farming method practiced on the control animal where the farming method was known and confirmed before the animal derived product was obtained from the control animal.

As used herein, a “methylated nucleotide” or a “methylated nucleotide base” refers to the presence of a methyl moiety on a nucleotide base, where the methyl moiety is usually not present in a recognized typical nucleotide base. For example, cytosine in its usual form does not contain a methyl moiety on its pyrimidine ring, but 5-methylcytosine contains a methyl moiety at position 5 of its pyrimidine ring. Therefore, cytosine in its usual form may not be considered a methylated nucleotide and 5-methylcytosine may be considered a methylated nucleotide. In another example, thymine may contain a methyl moiety at position 5 of its pyrimidine ring, however, for purposes herein, thymine may not be considered a methylated nucleotide when present in DNA. Typical nucleotide bases for DNA are thymine, adenine, cytosine and guanine. Typical bases for RNA are uracil, adenine, cytosine and guanine. Correspondingly a “methylation site” is the location in the target gene nucleic acid region where methylation has the possibility of occurring. For example, a location containing CpG is a methylation site wherein the cytosine may or may not be methylated. In particular, the term “methylated nucleotide” refers to nucleotides that carry a methyl group attached to a position of a nucleotide that is accessible for methylation. These methylated nucleotides are usually found in nature and to date, methylated cytosine that occurs mostly in the context of the dinucleotide CpG, but also in the context of CpNpG- and CpNpN-sequences may be considered the most common. In principle, other naturally occurring nucleotides may also be methylated but they will not be taken into consideration with regard to any aspect of the present invention.

As used herein, a “CpG site” or “methylation site” is a nucleotide within a nucleic acid (DNA or RNA) that is susceptible to methylation either by natural occurring events in vivo or by an event instituted to chemically methylate the nucleotide in vitro.

As used herein, a “methylated nucleic acid molecule” refers to a nucleic acid molecule that contains one or more nucleotides that is/are methylated.

The term ‘epigenetic change’ as used herein refers to a chemical (e.g., methylation) change or protein (e.g., histones) change that takes place to a gene body or a promoter thereof. Through epigenetic changes, environmental factors like. diet, stress and prenatal nutrition can make an imprint on genes passed from one generation to the next.

The term “bisulfite” as used herein encompasses any suitable type of bisulfite, such as sodium bisulfite, or another chemical agent that is capable of chemically converting a cytosine (C) to a uracil (U) without chemically modifying a methylated cytosine and therefore can be used to differentially modify a DNA sequence based on the methylation status of the DNA, e.g., U.S. Pat. Pub. US 2010/0112595 (Menchen et al.). As used herein, a reagent that “differentially modifies” methylated or non-methylated DNA encompasses any reagent that modifies methylated and/or unmethylated DNA in a process through which distinguishable products result from methylated and non-methylated DNA, thereby allowing the identification of the DNA methylation status. Such processes may include, but are not limited to, chemical reactions (such as a C to U conversion by bisulfite) and enzymatic treatment (such as cleavage by a methylation-dependent endonuclease). Thus, an enzyme that preferentially cleaves or digests methylated DNA is one capable of cleaving or digesting a DNA molecule at a much higher efficiency when the DNA is methylated, whereas an enzyme that preferentially cleaves or digests unmethylated DNA exhibits a significantly higher efficiency when the DNA is not methylated.

In context of the present invention also any “non-bisulfite-based method” and “non-bisulfite-based quantitative method” are comprised to test for a methylation status at any given methylation site to be tested. Such terms refer to any method for quantifying methylated or non-methylated nucleic acid that does not require the use of bisulfite. The terms also refer to methods for preparing a nucleic acid to be quantified that do not require bisulfite treatment. Examples of non-bisulfite-based methods include, but are not limited to, methods for digesting nucleic acid using one or more methylation sensitive enzymes and methods for separating nucleic acid using agents that bind nucleic acid based on methylation status. The terms “methyl-sensitive enzymes” and “methylation sensitive restriction enzymes” are DNA restriction endonucleases that are dependent on the methylation state of their DNA recognition site for activity. For example, there are methyl-sensitive enzymes that cleave or digest at their DNA recognition sequence only if it is not methylated. Thus, an unmethylated DNA sample will be cut into smaller fragments than a methylated DNA sample. Similarly, a hypermethylated DNA sample will not be cleaved. In contrast, there are methyl-sensitive enzymes that cleave at their DNA recognition sequence only if it is methylated. As used herein, the terms “cleave”, “cut” and “digest” are used interchangeably.

A “biological sample” in context of the invention may comprise any biological material obtained from the subject or group of subjects that contains genomic material, and may be liquid, solid or both, may be tissue or bone, or a body fluid such as blood, lymph, etc. In particular, the biological sample useful for the present invention may comprise biological cells or fragments thereof.

As used herein, the term “pre-selected methylation sites” refers to methylation sites that were selected from genes or regions that showed the highest degree of methylation variation during the training of the method and fulfils certain quality criteria such as a minimum sequencing coverage of ≥5× were considered and for ≥5 qualified CpG sites. Additionally, genes that have an average methylation level <0.1 or an average methylation level >0.9 can be excluded due to their limited dynamic range. “Reference methylation profiles” may be defined on the basis of multiple training samples using multivariate statistical methods, such as such as Principal Component analysis or Multi-Dimensional Scaling.

The term “pre-determined reference profile” used herein refers to a typical or standard methylation profile of the genomic material of a type of reference animal-derived product that is confirmed to be correctly labelled or certified. In one example, the pre-determined reference profile may be used in the context of a control animal, where the control animal has been correctly certified (i.e. the control animal has been bred according to a known distinct type of animal husbandry or farming method). In particular, the term “pre-determined reference profile” herein may be used in the context of a control animal, where the control animal has been correctly certified (i.e. based on the animal husbandry technique under which the control animal was bred or reared. The control animal may thus have been reared under Stable housing (Stallhaltung), Stable housing Plus (StallhaltungPlus), Outside climate (Auβenklima) or Premium (Premium) conditions. A panel of pre-determined reference profiles for control animals may also include profiles from different samples that have been obtained from different parts of control animals (animals reared from at least one, two, three or four of the animal husbandry conditions). For example, the panel of pre-determined reference profiles may include at least one profile for egg, at least one profile for meat (muscle, tissue, organs etc.), at least one profile for milk and the like. Each of these samples may have its own unique pre-determined methylation reference profile that also forms a part of the panel of pre-determined reference profiles. The panel may also include a pre-determined reference profile for each of these animal derived products specific for each of the four animal husbandry techniques.

A panel of pre-determined reference profiles may be prepared for different samples that are from animals that have been confirmed to be reared according to at least one of 2, 3, 4 or more animal husbandry techniques. Again here, there may be a panel of pre-determined reference profiles for each product that is derived from the animal that has been bred according to at least one of these four animal husbandry techniques. For example, the panel of pre-determined reference profiles may include at least one profile for egg, at least one profile for meat (muscle, tissue, organs etc.), at least one profile for milk and the like from animals that have been bred according to the animal husbandry that falls under the German Haltungsform category 1, Stable Housing (Stallhaltung). Each of these samples may have its own unique pre-determined methylation reference profile that also forms a part of the panel of pre-determined reference profiles. A second panel of pre-determined reference profiles may include at least one profile for egg, at least one profile for meat (muscle, tissue, organs etc.), at least one profile for milk and the like from animals that have been bred according to the animal husbandry that falls under the German Haltungsform category 2, Stable housing Plus (StallhaltungPlus). A third panel of pre-determined reference profiles may include at least one profile for egg, at least one profile for meat (muscle, tissue, organs etc.), at least one profile for milk and the like from animals that have been bred according to the animal husbandry that falls under the German Haltungsform category 3, Outside climate (Auβenklima). A fourth panel of pre-determined reference profiles may include at least one profile for egg, at least one profile for meat (muscle, tissue, organs etc.), at least one profile for milk and the like from animals that have been bred according to the animal husbandry that falls under the German Haltungsform category 4, Premium (Premium) conditions. In one example, the panel of pre-determined reference profiles may include all four different panels. In yet another example, the panel may be based on the different animal husbandry techniques found in a particular, land, state or geographical location. The number of panels of pre-determined reference profiles may vary depending on where the method is carried out and what the animal farming and/or animal husbandry techniques practiced in the country or region may be.

The methylation profile of different types of meat from animals grown under a particular animal husbandry technique may be identical. The methylation profile of different types of meat from different species of animals reared under particular animal husbandry technique may also be identical.

There may be a compilation of several pre-determined reference profiles and comparing the methylation profile of the test sample with the pre-determined reference profiles in the compilation may enable identifying the specific pre-determined reference profile that is (significantly) similar to the methylation profile of the test sample and then the test sample may be confirmed to have been reared under a distinct animal husbandry technique or not. In one example, the pre-determined reference profiles may include methylation profiles of different parts of meat (i.e. breast, thigh, kidney, liver, shoulder, ribs, intestines, etc.) from an animal (chicken, goat, cow, lamb, sheep etc.) that has been reared under a distinct animal husbandry technique.

In particular, the panel of predetermined reference methylation profiles according to any aspect of the present invention is distinct for different test animal-derived products. That is to say, each predetermined reference methylation profile is distinct for a single animal-derived product. The panel of predetermined reference methylation profiles may thus include many different predetermined reference methylation profiles from different parts of an animal or several animals of the same biological taxon as the test animal. There will also be different panels of predetermined reference methylation profiles for different animal taxon, and the relevant panel of predetermined reference methylation profiles unique for an animal taxon will depend on the animal taxon of the test animal.

The term “significantly similar” in context of the present disclosure, and in particular in context with the comparison of methylation profiles (such as the comparison between test profiles (from test subject(s) (i.e. sample X) and reference profiles) shall mean a similarity observed by statistical means (i.e. by using bioinformatics) and/or also by observation using the eye. A significant similarity is observed for example if a test profile overlaps with a reference profile that is defined by multiple training samples through multivariate statistical methods, such as Principal Component analysis or Multi-Dimensional Scaling. In particular, a test profile is significantly similar to the pre-determined reference profile if more than 50, 55, 60, 65, 70, 75, 80, 85, 90, 95, 96, 97, 98, 99% % of the methylation pattern/profile overlaps with that of the reference profile. A similarity of a test profile to more than one, such as two, three or even all reference profile reduces the significance of the similarity. Of course, the similarity of the test methylation profile to the reference methylation profile takes into consideration the experimental error that occurs in all methods.

The animal-derived product sample may be a single type of meat, different types of meat, a single part of a type of meat, different parts of a single type of meat or different parts of different types of meat. The sample may be from any biological entity having a DNA genome and DNA genome methylation. In particular, the methylation site is a CpG site. The biological entity may be any animal excluding a pig. In particular, the animal may be selected from the group consisting of chicken, lamb, camel, cow, goat, sheep, horse, donkey, turkey, duck, goose, quail, rabbit and mule. More in particular, the animal may be selected from the group consisting of cow, sheep, goat, camel, chicken, goose, duck and turkey. The term ‘meat’ herein may thus be understood to include chicken, lamb, beef, mutton, goat, camel, chicken, goose, duck, turkey and mixtures thereof.

The one or more pre-selected methylation sites in (a) are methylation sites associated with tissue specific gene expression, preferably wherein the pre-selected methylation sites are associated with gene expression of one distinct tissue.

The tissue may be selected from
(i) metabolic tissue such as gut tissue, said gut tissue preferably being ileum or jejunum,
(ii) muscular tissue,
(iii) skin tissue, and
(iv) organ tissue, said organ tissue preferably being hepatic and/or pancreatic tissue.

According to a further aspect of the present invention, there is provided certification of a test animal derived product sample, certifying that the test animal has been bred under a distinct type of animal husbandry, wherein the method for determining that the test animal was bred under the distinct type of animal husbandry comprises the steps of:
(a) determining a test methylation profile of one or more pre-selected methylation sites within the genomic material obtained from the test animal derived product sample; and
(b) comparing the test methylation profile obtained from (a) with a panel of predetermined reference methylation profiles of the same biological taxon of the test animal from which the product sample derives, wherein each of the predetermined reference methylation profiles from the panel is a reference methylation profile of a control animal that has been bred under a known distinct type of animal husbandry,
wherein if the test methylation profile of (a) is significantly similar to one of the predetermined reference methylation profiles, it is indicative of the test animal having been bred under the same distinct type of animal husbandry as the control animal from which the reference methylation profile originates from and the test animal-derived product is certified so; and wherein the test animal is selected from livestock or poultry.

According to yet a further aspect of the present invention, there is provided a method of verifying certification of a test animal-derived product sample that has been certified to derive from a test animal that has been bred under a distinct type of animal husbandry, the method comprising the steps of:
(a) determining a test methylation profile of one or more pre-selected methylation sites within the genomic material obtained from the test animal derived product sample; and
(b) comparing the test methylation profile obtained from (a) with a reference methylation profile obtained from a control animal of the same biological taxon of the test animal from which the product sample is derived from, where the control animal is bred under the distinct type of animal husbandry that the test animal has been certified to be bred under,
wherein a significant similarity in the test methylation profile of (a) compared to the reference methylation profile from the control animal, is indicative of the test animal having been bred under the same distinct type of animal husbandry as the control animal and the test animal-derived product being correctly certified, and wherein a difference in the test methylation profile of (a) compared to the reference methylation profile of the control animal, is indicative of the test animal not having been bred under the same distinct type of animal husbandry as the control animal and the test animal-derived product being wrongly certified;
wherein the pre-selected methylation sites are CpG sites selected from genes or regions of genomic DNA from the control and test animal that show the highest degree of methylation variation during the training of the method; and wherein the test methylation profile has significant similarity to the reference methylation profile when the test methylation profile overlaps with the reference methylation profile that is defined by multiple training samples using Principal Component analysis and/or Multi-Dimensional Scaling; and wherein the methylation profile is determined using at least one method selected from the group consisting of PCR, methylation-specific PCR, real-time methylation-specific PCR, PCR assay using a methylation DNA-specific binding protein, quantitative PCR, a DNA chip-based assay, pyrosequencing, bisulfate pyrosequencing, Methylated DNA immunoprecipitation-sequencing and combinations thereof; and wherein the test animal is selected from livestock or poultry.

According to a further aspect of the present invention, there is provided a method of determining if a test animal-derived product sample, is obtained from a test animal that has been bred under a distinct type of animal husbandry, the method comprising:
comparison of a test methylation profile obtained from genomic material of the test animal derived product sample with a reference methylation profile obtained from a control animal of the same biological taxon of the test animal from which the product sample is derived from, where the control animal is has been bred under a known distinct type of animal husbandry; and
wherein the test animal is selected from livestock or poultry.

The method according to this aspect of the present invention, comprises the steps of:
a) determining the test methylation profile of one or more pre-selected methylation sites within the genomic material obtained from the test animal derived product sample; and
b) comparing the test methylation profile determined in (a) with a panel of predetermined reference methylation profiles of the same biological taxon of the test animal from which the product sample derives, where each predetermined reference methylation profile from the panel is a reference methylation of a control animal that has been bred under a known distinct type of animal husbandry,
wherein a significant similarity of in the test methylation profile of (a) compared to any one of the reference methylation profiles of the panel from the control animal, is indicative of the test animal having been bred under the same distinct type of animal husbandry as the control animal from which the reference methylation profile originates from; and wherein a difference in the test methylation profile of (a) compared to the reference methylation profile of the control animal, is indicative of the test animal having been bred under another distinct type of animal husbandry as the control animal; and wherein the pre-selected methylation sites are CpG sites selected from genes or regions of genomic DNA from the control and test animal that show the highest degree of methylation variation during the training of the method; and wherein the test methylation profile has significant similarity to the reference methylation profile when the test methylation profile overlaps with the reference methylation profile that is defined by multiple training samples using Principal Component analysis and/or Multi-Dimensional Scaling; and wherein the methylation profile is determined using at least one method selected from the group consisting of PCR, methylation-specific PCR, real-time methylation-specific PCR, PCR assay using a methylation DNA-specific binding protein, quantitative PCR, a DNA chip-based assay, pyrosequencing, bisulfate pyrosequencing, Methylated DNA immunoprecipitation-sequencing and combinations thereof; and wherein the test animal is selected from livestock or poultry.

The method a DNA chip-based assay refers to a DNA methylation based assay that may be carried out on a chip. For example, a bead based chip.

In particular, the difference according to any aspect of the present invention refer to a difference in methylation and is hypomethylation or hypermethylation.

In particular, the one or more pre-selected methylation sites according to any aspect of the present invention is selected from the list of CpG sites in Tables 2a and 2b.

According to yet another aspect of the present invention, there is provided a use of DNA methylation profiling for certifying of an animal-derived product sample, wherein the certification of the animal-derived product sample is based on the animal having been bred under a distinct type of animal husbandry and wherein the DNA methylation profiling includes the steps of:
(a) determining a test methylation profile of one or more pre-selected methylation sites within the genomic material obtained from the test animal derived product sample; and
(b) comparing the test methylation profile obtained from (a) with a reference methylation profile obtained from a control animal of the same biological taxon of the test animal from which the product sample is derived from, where the control animal is bred under a known distinct type of animal husbandry, and wherein the test animal is selected from livestock or poultry.

More in particular, the distinct type of animal husbandry is selected from the group consisting of:
Certified Standards, Stable housing (Stallhaltung), or equivalents thereof;
Enhanced Welfare, Stable housing Plus (StallhaltungPlus) or equivalents thereof;
Free Range, Outside climate (Auβenklima), Premium (Premium) or equivalents thereof; and
organic.

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

### Wet-Lab Methodology

Broiler chicken breast meat was obtained from three different German supermarkets to obtain replicate samples of as many of the German certification standards, known as Haltungsform, as possible. There were four distinct Haltungsform categories for broiler chicken meat in Germany which include Haltungsform 1 (Stallhltung), 2 (Stallhaltung Plus), 3 (Auβenklima) and 4 (Premium). From Haltungsform 1-4 the rearing conditions were improving in alignment with assessed animal welfare needs. For example, space requirements increase from maximum 39 kg of chicken/m2 in Stallhaltung to 21 kg/m2 in the Premium category. Additional requirements defining each Haltungsform category include the genetic lines of the broiler chickens, the length of rearing (at last 81 days in Haltungsform 3 and 4), the amount of enrichments and outdoor access available as well as the types of ingredients fed to the animals.

From this sample collection 2 replicate samples of Haltungsform 2 chicken breast meat from each of 3 grocery stores for a total of 6 replicates were obtained, 3 replicate samples of Haltungsform 2 chicken breast meat from 1 grocery store was obtained and 3 replicate samples of Haltungsform 4 chicken breast meat samples from 1 grocery store were obtained. Unfortunately, it was not possible to obtain clearly labelled Haltungsform 1 samples, so this category was excluded. In total there were 12 samples covering 3 of the 4 available Haltungsform categorisations (Table 1).

TABLE 1
Sample identification of the 12 chicken breast meat samples obtained
from 3 of the 4 distinct Haltungsform rearing conditions.

| | se- | | | | |
|---|---|---|---|---|---|
| | quencing | | | | |
| DNA | sample | | Super- | Rearing | repli- |
| sample id | name | Type | market | condition | cate |
| CHKN-0015 | SR_15 | Frozen | Aldi | Haltungsform 2 | Sample 1 |
| CHKN-0016 | SR_16 | Frozen | Aldi | Haltungsform 2 | Sample 2 |
| CHKN-0018 | SR_18 | Frozen | Lidl | Haltungsform 2 | Sample 1 |
| CHKN-0019 | SR_19 | Frozen | Lidl | Haltungsform 2 | Sample 2 |
| CHKN-0021 | SR_21 | Frozen | Rewe | Haltungsform 2 | Sample 1 |
| CHKN-0022 | SR_22 | Frozen | Rewe | Haltungsform 2 | Sample 2 |
| CHKN-0024 | SR_24 | Frozen | Aldi | Haltungsform 3 | Sample 1 |
| CHKN-0025 | SR_25 | Frozen | Aldi | Haltungsform 3 | Sample 2 |
| CHKN-0026 | SR_26 | Frozen | Aldi | Haltungsform 3 | Sample 3 |
| CHKN-0027 | SR_27 | Frozen | Lidl | Haltungsform 4 | Sample 1 |
| CHKN-0028 | SR_28 | Frozen | Lidl | Haltungsform 4 | Sample 2 |
| CHKN-0029 | SR_29 | Frozen | Lidl | Haltungsform 4 | Sample 3 |

Genomic DNA was purified from the breast tissue samples using the DNeasy Blood & Tissue Kit (Qiagen) and is quantified using the PicroGreen or NanoDrop™ 2000.

The genomic DNA (500 ng) from breast tissue samples were used to prepare libraries for Whole Genome Bisulfite Sequencing (WGBS). The sequencing of the libraries was performed by a third party on a NovaSeq platform which generated 125 GB data per sample with 20X coverage.

### Computational Methodology

### Processing:

Sequenced reads were trimmed and mapped with BSMAP¹ version 2.5 using the assembly version 5.0 of the chicken (Gallus gallus) genome as reference sequence. After deduplication using picard², the methylation ratios were determined using a Python script (methratio.py) distributed with the BSMAP package. For all further analysis, only CpGs covered by at least ten reads were considered, which resulted in 6458063 CpG sites.

### Differential Methylation Analysis

Differential methylation analysis was performed using MethylKit3 (version 1.12.0) between the different Haltungsform group. MethylKit uses logistic regression to calculate p-values and sliding linear model method⁴ to adjust the p-values to q-values.

CpG sites with an FDR below 0.05 and a methylation change larger than 25% between the groups were considered as significantly differentially methylated Positions (DMPs), resulting in 201246 CpG sites. An example of the CpG sites is provided in Table 2.

methylKit generates a ‘prcomp’ object, which can be used to extract and plot the principal components. Principle Component Analysis (PCA) is a dimensionality-reduction method which can transform large data sets to a few principal components. The first few principal components generally retain most of the variation present in the dataset and are useful for emphasizing the grouping structure in the data

### Results

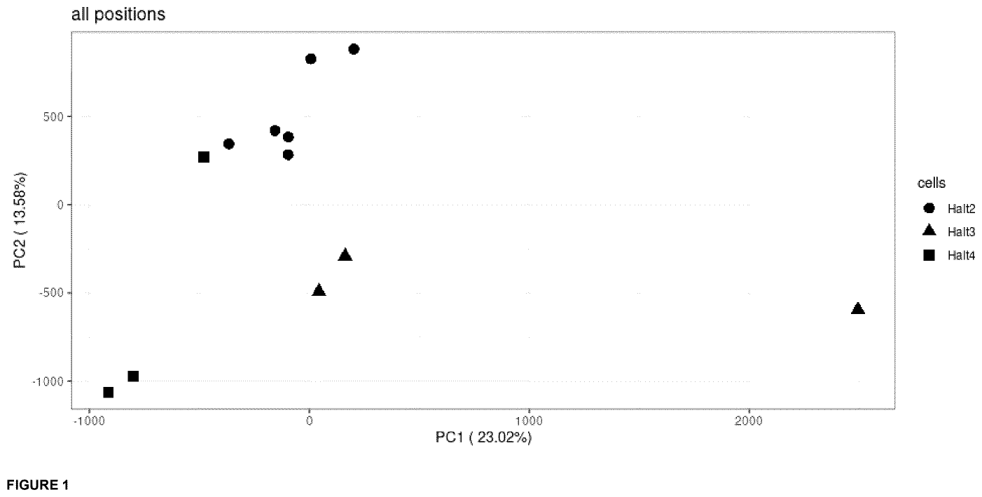

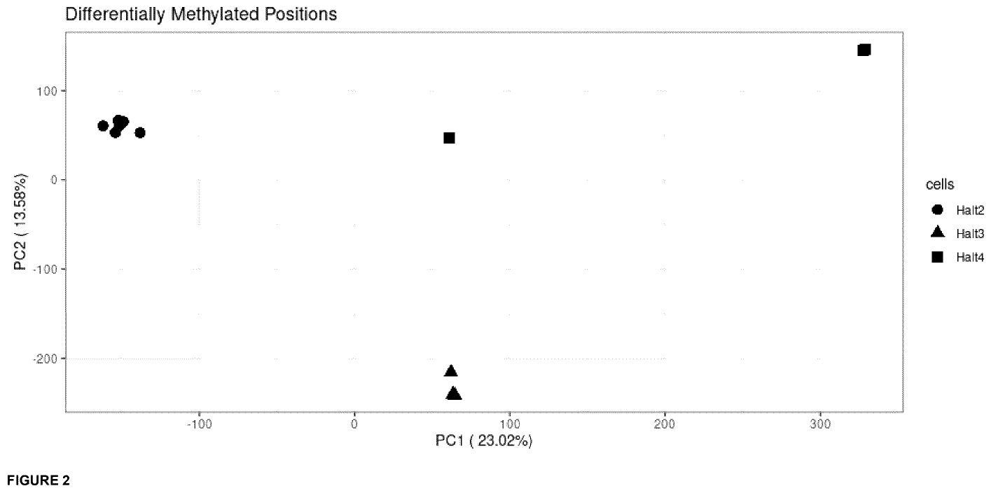

The PCA of all samples using CpG sites with a minimum coverage of 10 for all 12 samples showed distinct clusterings based on Haltungsform otherwise known as rearing condition (FIG. 1). When a second PCA was run using the differentially methylated positions this clustering based on Haltungsform, otherwise known as rearing condition, became even clearer (FIG. 2).

In summary, plotting of the first two principal components of the CpGs before and after differential methylation analysis reveal meaningful clustering of the samples. Differentially methylated Positions (DMPs) i.e. CpG sites were able to effectively cluster of the different groups in a meaningful way. Specific CpG sites related to rearing condition categorisations were determined and allow for the creation of reference profiles for future assessment of chicken meat samples for determination of rearing category.

TABLE 2a
Representative CpG sites from the chicken genome that were differentially methylated
in breast meat DNA from chickens in 3 different Haltungsform rearing categories.
Table shows an example 504 of the total chicken environmental specific CpG sites.

| Chrom. 1 | Chrom. 2 | Chrom 3. | Chrom 4. | Chrom. 5 | Chrom. 6 | Chrom. 7 | Chrom. 8 |
|---|---|---|---|---|---|---|---|
| 48320135 | 91506296 | 35314037 | 65711658 | 53367140 | 25431491 | 33283167 | 22475003 |
| 30656200 | 75956409 | 9663346 | 595174 | 40475620 | 31731812 | 10865253 | 19767958 |
| 72019344 | 107770668 | 45058374 | 65690473 | 23766287 | 33370556 | 36230362 | 12562918 |
| 106294434 | 45707148 | 97887288 | 85040903 | 39322420 | 16327741 | 10778771 | 27261388 |
| 66901855 | 142246141 | 27694382 | 29564906 | 5790293 | 12476024 | 30016688 | 8015446 |
| 49035885 | 84186239 | 49568466 | 16465102 | 17338183 | 19830112 | 36120386 | 24043289 |
| 179635732 | 5963815 | 68934780 | 48304338 | 17913085 | 11181698 | 17573342 | 20590064 |
| 68523581 | 35013461 | 35332935 | 55242298 | 17338207 | 20205150 | 1243408 | 27208366 |
| 50866176 | 16383809 | 40066489 | 8266689 | 44729484 | 24095468 | 3218471 | 16458371 |
| 146031733 | 40549925 | 4769729 | 69056036 | 53391620 | 16214005 | 27996886 | 22528660 |
| 23437176 | 81412635 | 97678639 | 88457365 | 9130825 | 9647629 | 19230823 | 16540199 |
| 63156586 | 115042415 | 31241293 | 88202375 | 46244051 | 16993318 | 14890448 | 20943212 |
| 66905900 | 75949903 | 80796001 | 91194373 | 8515082 | 12468000 | 17383700 | 4443865 |
| 23485779 | 97175898 | 9660888 | 39205675 | 53363620 | 11142869 | 3228320 | 4072729 |
| 124331834 | 81264519 | 52604248 | 79202255 | 15250170 | 24096254 | 19230886 | 22474022 |
| 50906457 | 38700361 | 104977379 | 54530640 | 16227895 | 24632200 | 11643438 | 16464252 |
| 30855766 | 80683670 | 52604907 | 34539405 | 8440478 | 21198435 | 5136835 | 22659182 |
| 23499431 | 84086313 | 7952160 | 10970476 | 41593575 | 11178375 | 13626512 | 19320991 |
| 44716565 | 40897513 | 80593781 | 90536087 | 9042633 | 25669294 | 25390853 | 17037414 |
| 174522696 | 44949951 | 88111952 | 3901112 | 43567016 | 25484417 | 28372647 | 25962806 |
| 27177802 | 139953859 | 82662107 | 13943722 | 53377206 | 24618352 | 3601015 | 17331357 |
| 13730924 | 91505832 | 80752038 | 13716534 | 19255641 | 6663510 | 10865246 | 16759136 |
| 128165785 | 4344592 | 38309980 | 85215583 | 37737264 | 30601723 | 34409735 | 20793056 |
| 125401749 | 127700034 | 52604057 | 55387733 | 42762964 | 33456355 | 29921595 | 16446059 |
| 51295869 | 83048060 | 105182430 | 85050818 | 35899080 | 24186453 | 17308548 | 16528414 |
| 28437676 | 81340475 | 27362714 | 54433137 | 46087465 | 31106726 | 10549103 | 20397652 |
| 107830140 | 97419181 | 39750440 | 55207246 | 9159388 | 25636911 | 31546286 | 3889117 |
| 168773184 | 81257477 | 3637916 | 12870694 | 17254875 | 32040392 | 3288786 | 25537766 |
| 165708998 | 139965997 | 4765946 | 16436699 | 46352871 | 9602293 | 19284993 | 22638426 |
| 30710630 | 48850331 | 108623109 | 54967198 | 9175529 | 16885445 | 301052 | 16742497 |
| 175324262 | 64765026 | 7703851 | 79182458 | 9043552 | 12338854 | 11134317 | 2359810 |
| 13740250 | 139823802 | 97621257 | 7035179 | 19665193 | 32014676 | 33185188 | 25588582 |
| 107124790 | 97175988 | 39328498 | 10550654 | 22818782 | 21671795 | 10540454 | 4053598 |
| 179636348 | 118045429 | 26921255 | 62190780 | 14301451 | 31745879 | 19336408 | 26812538 |
| 28416758 | 103166693 | 58333962 | 57539608 | 54965840 | 33452549 | 17313031 | 8884574 |
| 69136728 | 20126945 | 38318324 | 2326350 | 18224331 | 16342370 | 3197832 | 28270707 |

| Chrom. 9 | Chrom. 10 | Chrom. 11 | Chrom. 12 | Chrom. 13 | Chrom. 14 |
|---|---|---|---|---|---|
| 19665110 | 1209634 | 16297666 | 16262168 | 15756413 | 12846736 |
| 9515502 | 3396777 | 16333906 | 3522090 | 10486727 | 10424689 |
| 5954179 | 16689870 | 15104459 | 6969685 | 9786827 | 6763515 |
| 5893929 | 1213600 | 16528362 | 11171838 | 4405377 | 6380872 |
| 9235308 | 2248147 | 8153807 | 13914650 | 17034057 | 12902179 |
| 14616688 | 2643474 | 15854193 | 13700466 | 16525353 | 2086895 |
| 14649112 | 18224155 | 15707192 | 11313089 | 2799936 | 6263600 |
| 14443864 | 8022733 | 16793888 | 10673026 | 17330579 | 11797926 |
| 4280573 | 4848653 | 15727141 | 13680629 | 16645756 | 12844669 |
| 14447298 | 16395790 | 18101330 | 9031586 | 13395656 | 7037431 |
| 14447280 | 2646085 | 7682673 | 18694275 | 4063633 | 7872635 |
| 4856047 | 11934350 | 15230284 | 18691997 | 4347238 | 6695621 |
| 1460901 | 9692390 | 7646291 | 14113348 | 6788566 | 12024091 |
| 1209337 | 11326963 | 15912338 | 13723574 | 17173391 | 12026983 |
| 12395813 | 13794512 | 15826926 | 6801745 | 3238696 | 12824895 |
| 14572215 | 1357842 | 18553536 | 18514642 | 8232169 | 12025940 |
| 11989297 | 9717764 | 8216208 | 15606155 | 7160462 | 10667748 |
| 2441030 | 2019889 | 10194554 | 11520596 | 10307819 | 7834956 |
| 14794034 | 1638208 | 15675006 | 6804530 | 4343408 | 6393294 |
| 14616621 | 1909185 | 17983450 | 9315739 | 3337726 | 2018561 |
| 9498792 | 4531626 | 15613619 | 17690516 | 15723163 | 7871547 |
| 14190671 | 18434522 | 15816085 | 19592539 | 9227019 | 12016846 |
| 20237396 | 3435740 | 8039839 | 1411559 | 13601612 | 8208138 |
| 12173377 | 19429472 | 15907658 | 16322825 | 9101292 | 2382045 |
| 14452873 | 5273686 | 18556669 | 14129589 | 17020552 | 8185696 |
| 4917489 | 6482521 | 470054 | 5529630 | 16124272 | 7838439 |
| 14582031 | 14048634 | 15909213 | 19282853 | 13186492 | 11817943 |
| 12626724 | 19228285 | 15364413 | 11618476 | 7860599 | 8048066 |
| 19151294 | 16820229 | 17079775 | 1181719 | 16529582 | 6031755 |
| 1131790 | 8664876 | 6521729 | 10387493 | 17190037 | 4198723 |
| 3313912 | 18279260 | 13463071 | 11879260 | 13519450 | 14454672 |
| 12098351 | 8666816 | 4076803 | 11844199 | 10835365 | 2366280 |
| 13294175 | 2583455 | 10349844 | 9317306 | 3337245 | 5176768 |
| 15614531 | 18472616 | 8515291 | 14002631 | 16987221 | 12827625 |
| 14105780 | 5278046 | 18733895 | 1784254 | 12891369 | 12837592 |
| 19079417 | 1986822 | 3944602 | 9827747 | 10371451 | 12525590 |

TABLE 2b
Representative CpG sites from the chicken genome that were differentially methylated
in breast meat DNA from chickens in 3 different Haltungsform rearing categories.
Table shows an example 458 of the total chicken environmental specific CpG sites.

| Chrom. 15 | Chrom. 16 | Chrom. 17 | Chrom. 18 | Chrom. 19 | Chrom. 20 | Chrom. 21 | Chrom. 22 |
|---|---|---|---|---|---|---|---|
| 1188983 | 80158 | 1691724 | 3634694 | 8019295 | 6940061 | 4110057 | 3847387 |
| 7419566 | 52906 | 10260540 | 8879025 | 5360821 | 8140184 | 6555537 | 4077856 |
| 6455898 | 91218 | 10056026 | 2840458 | 1984530 | 6551287 | 6571408 | 2908068 |
| 1693116 | 48677 | 6089857 | 9604134 | 1839532 | 5379720 | 2286401 | 4075529 |
| 1613596 | 172834 | 6089887 | 3648284 | 2882010 | 10211866 | 6612220 | 2898631 |
| 10375536 | 95107 | 9790371 | 3060593 | 5248091 | 11521548 | 5348574 | 4426965 |
| 1658136 | 11327 | 9848804 | 2451973 | 1981945 | 10251559 | 5429210 | 1519490 |
| 1641767 | 180847 | 4990295 | 7371436 | 7342048 | 6610374 | 910218 | 2296649 |
| 5245928 | 206292 | 10140572 | 791953 | 8931516 | 6566509 | 4096655 | 4166845 |
| 1611781 | 115070 | 9156210 | 9583270 | 5351210 | 7214804 | 2507664 | 2908058 |
| 2146170 | 95555 | 7954595 | 2952619 | 5708093 | 6715793 | 3131430 | 2775566 |
| 1619484 | 91230 | 10288286 | 8888732 | 1979016 | 4999880 | 430605 | 4487027 |
| 1775708 | | 9446019 | 10313333 | 2477648 | 5075299 | 3951522 | 4097824 |
| 1704207 | | 9604566 | 7640613 | 5358918 | 10680695 | 4649928 | 2298091 |
| 2662515 | | 7049510 | 9591158 | 395930 | 10250163 | 862040 | 4143053 |
| 1352865 | | 9871986 | 9584859 | 7665847 | 13739833 | 3541489 | 4139431 |
| 2229555 | | 10494842 | 3546989 | 6510293 | 5777725 | 4718077 | 2888761 |
| 9763212 | | 10067401 | 3677052 | 3264065 | 6047217 | 5564895 | 4371974 |
| 1780406 | | 10267555 | 6451255 | 7281367 | 5085891 | 401072 | 4377555 |
| 11693696 | | 10236256 | 1509938 | 1938049 | 7560261 | 440846 | 4073042 |
| 1850827 | | 9921622 | 3810801 | 7209175 | 6940250 | 6295782 | 2951207 |
| 1614244 | | 3725965 | 3032106 | 8004036 | 6013467 | 4707388 | 1692659 |
| 5172108 | | 10088808 | 4986939 | 5111485 | 6651135 | 6555979 | 3755938 |
| 7507178 | | 6095364 | 2316397 | 7412187 | 10539557 | 3012957 | |
| 594360 | | 1293675 | 3333424 | 5839872 | 6574439 | 1123720 | |
| 1680794 | | 10252387 | 959481 | 1003675 | 5584808 | 3548193 | |
| 5661199 | | 5754259 | 3817216 | 2582469 | 6563989 | 5340786 | |
| 12399813 | | 10236351 | 2600615 | 6374694 | 8489647 | 5432862 | |
| 7514741 | | 9826196 | 8942920 | 7242371 | 10592983 | 5025841 | |
| 5176048 | | 8658249 | 3582719 | 1801021 | 10120694 | 6734739 | |
| 5191232 | | 9826087 | 2769434 | 395961 | 10539518 | 4200278 | |
| 6937795 | | 10146064 | 827597 | 3245772 | 5572926 | 6656616 | |
| 1504622 | | 1265338 | 2243323 | 2474945 | 9161468 | 4115389 | |
| 11515698 | | 10924176 | 3794274 | 7405606 | 5301859 | 2863582 | |
| 10175649 | | 9810724 | 3700563 | 5584577 | 4759569 | 3910471 | |
| 3680557 | | 3896706 | 3800308 | 5068108 | 6470699 | 5047549 | |
| | Chrom. 23 | Chrom. 24 | Chrom. 25 | Chrom. 26 | Chrom. 27 | Chrom. 28 | Chrom. 33 |
| | 1681614 | 3069169 | 1820248 | 2614007 | 4132922 | 1859733 | 162591 |
| | 1560418 | 1722735 | 269344 | 2605593 | 1361291 | 3373394 | 957099 |
| | 1843911 | 1167177 | 257246 | 846876 | 4818260 | 2904126 | 165114 |
| | 170929 | 5415362 | 249454 | 2393809 | 1582033 | 1069143 | 599154 |
| | 3980965 | 1712034 | 241246 | 4881555 | 2493235 | 1577997 | 1382127 |
| | 5041611 | 1210072 | 1148851 | 851729 | 1273378 | 2079407 | 599116 |
| | 1560353 | 1178590 | 1857344 | 3736575 | 2730901 | 3218402 | 110341 |
| | 3752838 | 5406346 | 8868 | 4955290 | 1241594 | 1510646 | 700505 |
| | 1671853 | 727961 | 806227 | 2253771 | 2147598 | 3468498 | |
| | 3133406 | 5403519 | 2153603 | 2635822 | 4483942 | 4646583 | |
| | 616284 | 5472723 | 168908 | 2105599 | 1444638 | 661354 | |
| | 2972163 | 1171840 | 2760819 | 805284 | 1817333 | 3635463 | |
| | 3698378 | 1198031 | 1477026 | 4883462 | 3110991 | 3635468 | |
| | 4396812 | 3356183 | 1873556 | 4532514 | 4815397 | 4149610 | |
| | 1563165 | 2975525 | 932869 | 3218758 | 1432737 | 4119291 | |
| | 2892700 | 480771 | 2139925 | 3487761 | 4925353 | 4063148 | |
| | 4155062 | 4317709 | 914317 | 3852099 | 1591385 | 4403855 | |
| | 3455603 | 1554408 | 1857307 | 1927056 | 1446116 | 3408910 | |
| | 1644770 | 3235804 | 249393 | 4140717 | 4722928 | 2480439 | |
| | 1358383 | 3405343 | | 3811629 | 1272954 | 1768357 | |
| | 3956350 | 1728395 | | 3525140 | 2928012 | 3159781 | |
| | 1081276 | 4740204 | | 2626329 | 1607807 | 3373382 | |
| | 1447872 | 4308944 | | 2326051 | 193969 | 1820509 | |
| | 1495054 | 1962938 | | 3861586 | 3224595 | 3457529 | |
| | 1662378 | 5522036 | | 3835724 | 1414567 | 1820393 | |
| | 5140971 | 695026 | | 4526784 | 273820 | 2032523 | |
| | 2954441 | 1924317 | | 2407150 | 4932995 | 1584365 | |
| | 3687553 | 5659486 | | 3670126 | 1306791 | 2082744 | |
| | 2788753 | 4264381 | | 4017382 | 3654731 | 2465650 | |
| | 2804218 | 1592074 | | 3824402 | 1770333 | 2074358 | |
| | 1727762 | 1752440 | | 3827146 | 3235638 | 1166091 | |
| | 3119414 | 4666582 | | 4021562 | 1274407 | 3582224 | |
| | 1966144 | 648004 | | 2876593 | 3237594 | 1430424 | |
| | 2975087 | 4427177 | | 2971790 | 3169645 | 4066751 | |
| | 4140867 | 1845001 | | 5112841 | 1234037 | 4592882 | |
| | 1270875 | 6014351 | | 2743078 | 2966444 | 530997 | |

1. A method of certifying a test animal-derived product sample, the method comprising the steps of:
(a) determining a test methylation profile of one or more pre-selected methylation sites within the genomic material obtained from the test animal derived product sample; and
(b) comparing the test methylation profile obtained from (a) with a reference methylation profile obtained from a control animal of the same biological taxon of the test animal from which the product sample is derived from, where the control animal is bred under a known distinct type of animal husbandry,
wherein a significant similarity in the test methylation profile of (a) compared to the reference methylation profile from the control animal, is indicative of the test animal having been bred under the same distinct type of animal husbandry as the control animal and the test animal-derived product is certified so; and
wherein a difference in the test methylation profile of (a) compared to the reference methylation profile of the control animal, is indicative of the test animal having been bred under another distinct type of animal husbandry as the control animal; and
wherein the pre-selected methylation sites are CpG sites selected from genes or regions of genomic DNA from the control and test animal that show the highest degree of methylation variation during the training of the method; and
wherein the test methylation profile has significant similarity to the reference methylation profile when the test methylation profile overlaps with the reference methylation profile that is defined by multiple training samples using Principal Component analysis and/or Multi-Dimensional Scaling; and
wherein the methylation profiles are determined using at least one method selected from the group consisting of PCR, methylation-specific PCR, real-time methylation-specific PCR, PCR assay using a methylation DNA-specific binding protein, quantitative PCR, a DNA chip-based assay, pyrosequencing, bisulfate pyrosequencing, Methylated DNA immunoprecipitation-sequencing and combinations thereof; and
wherein the test animal is selected from livestock or poultry.

2. The method according to claim 1, wherein in step (b),
the test methylation profile determined in (a) is compared with a panel of predetermined reference methylation profiles of the same biological taxon of the test animal from which the product sample derives, wherein each of the predetermined reference methylation profiles is specific for a control animal that has been bred under a known distinct type of animal husbandry, and
wherein if the test methylation profile is significantly similar to one of the predetermined reference methylation profiles, the test animal derived product sample has the same distinct certification as that of the control animal from which the predetermined reference methylation profile was obtained and the test animal-derived product is certified so.

3. A method of verifying certification of a test animal-derived product sample that has been certified to derive from a test animal that has been bred under a distinct type of animal husbandry, the method comprising the steps of:
(a) determining a test methylation profile of one or more pre-selected methylation sites within the genomic material obtained from the test animal derived product sample; and
(b) comparing the test methylation profile obtained from (a) with a reference methylation profile obtained from a control animal of the same biological taxon of the test animal from which the product sample is derived from, where the control animal is bred under the distinct type of animal husbandry that the test animal has been certified to be bred under,
wherein a significant similarity in the test methylation profile of (a) compared to the reference methylation profile from the control animal, is indicative of the test animal having been bred under the same distinct type of animal husbandry as the control animal and the test animal-derived product being correctly certified, and
wherein a difference in the test methylation profile of (a) compared to the reference methylation profile of the control animal, is indicative of the test animal not having been bred under the same distinct type of animal husbandry as the control animal and the test animal-derived product being wrongly certified;
wherein the pre-selected methylation sites are CpG sites selected from genes or regions of genomic DNA from the control and test animal that show the highest degree of methylation variation during the training of the method; and
wherein the test methylation profile has significant similarity to the reference methylation profile when the test methylation profile overlaps with the reference methylation profile that is defined by multiple training samples using Principal Component analysis and/or Multi-Dimensional Scaling; and
wherein the methylation profile is determined using at least one method selected from the group consisting of PCR, methylation-specific PCR, real-time methylation-specific PCR, PCR assay using a methylation DNA-specific binding protein, quantitative PCR, a DNA chip-based assay, pyrosequencing, bisulfate pyrosequencing, Methylated DNA immunoprecipitation-sequencing and combinations thereof;
and
wherein the test animal is selected from livestock or poultry.

4. A method of determining if a test animal-derived product sample, is obtained from a test animal that has been bred under a distinct type of animal husbandry, the method comprising the steps of:
a) determining the test methylation profile of one or more pre-selected methylation sites within the genomic material obtained from the test animal derived product sample;
and
b) comparing the test methylation profile determined in (a) with a panel of predetermined reference methylation profiles of the same biological taxon of the test animal from which the product sample derives, where each predetermined reference methylation profile from the panel is a reference methylation of a control animal that has been bred under a known distinct type of animal husbandry,
wherein a significant similarity of in the test methylation profile of (a) compared to any one of the reference methylation profiles of the panel from the control animal, is indicative of the test animal having been bred under the same distinct type of animal husbandry as the control animal from which the reference methylation profile originates from
wherein a difference in the test methylation profile of (a) compared to the reference methylation profile of the control animal, is indicative of the test animal having been bred under another distinct type of animal husbandry as the control animal; and
wherein the pre-selected methylation sites are CpG sites selected from genes or regions of genomic DNA from the control and test animal that show the highest degree of methylation variation during the training of the method; and
wherein the test methylation profile has significant similarity to the reference methylation profile when the test methylation profile overlaps with the reference methylation profile that is defined by multiple training samples using Principal Component analysis and/or Multi-Dimensional Scaling; and
wherein the methylation profile is determined using at least one method selected from the group consisting of PCR, methylation-specific PCR, real-time methylation-specific PCR, PCR assay using a methylation DNA-specific binding protein, quantitative PCR, a DNA chip-based assay, pyrosequencing, bisulfate pyrosequencing, Methylated DNA immunoprecipitation-sequencing and combinations thereof;
and
wherein the test animal is selected from livestock or poultry.

5. The method according to claim 1, wherein the distinct type of animal husbandry under which the control and/or test animal is bred is selected from the group consisting at least of:
Certified Standards, Stable housing (Stallhaltung), or equivalents thereof;
Enhanced Welfare, Stable housing Plus (StallhaltungPlus) or equivalents thereof;
Free Range, Outside climate (Auβenklima), Premium (Premium) or equivalents thereof; and
organic.

6. The method according to claim 1, wherein the livestock includes terrestrial and aquatic livestock.

7. The method according to claim 6, wherein the aquatic livestock is selected from the group consisting of carps, salmonids, tilapias, catfish, marine and brackish fishes, soft-shelled turtles, barramundi, marine shrimp, mitten crabs, marbled crayfish and other decapod crustaceans, bivalves, gastropods, and the terrestrial livestock is selected from the group consisting of cow, goat, sheep, pig, horse, donkey, rabbit and mule and/or poultry which is selected from the group consisting of chicken, turkey, duck, goose, and quail.

8. The method according to claim 1, wherein the animal-derived product is meat, muscle, at least one organ, milk, collagen, feather, blood and/or bone.

9. The method according to claim 1, wherein the one or more pre-selected methylation sites in (a) are methylation sites associated with tissue specific gene expression, preferably wherein the pre-selected methylation sites are associated with gene expression of one distinct tissue.

10. The method according to claim 1, wherein the one or more pre-selected methylation sites is selected from the list of CpG sites in Tables 2a and 2b.

11. Use of DNA methylation profiling for certifying of an animal-derived product sample, wherein the certification of the animal-derived product sample is based on the animal having been bred under a distinct type of animal husbandry and wherein the DNA methylation profiling includes the steps of:
(a) determining a test methylation profile of one or more pre-selected methylation sites within the genomic material obtained from the test animal derived product sample; and
(b) comparing the test methylation profile obtained from (a) with a reference methylation profile obtained from a control animal of the same biological taxon of the test animal from which the product sample is derived from, where the control animal is bred under a known distinct type of animal husbandry, and
wherein the test animal is selected from livestock or poultry.

12. Use according to claim 11, wherein the distinct types of animal husbandry under which the control and/or test animal is bred is selected from the group consisting of at least:
Certified Standards, Stable housing (Stallhaltung), or equivalents thereof;
Enhanced Welfare, Stable housing Plus (StallhaltungPlus) or equivalents thereof;
Free Range, Outside climate (Auβenklima), Premium (Premium) or equivalents thereof; and
organic.
